(19) United States
(12) Patent Application Publication
(10) Pub. No.: US 2018/0059015 A1
(43) Pub. Date: Mar. 1, 2018

(54) PERSONAL LIQUID ANALYSIS SYSTEM

(71) Applicant: Sensii, Inc., Cambridge, MA (US)

(72) Inventors: Qiaochu Li, Cambridge, MA (US); Wenting Xing, Cambridge, MA (US)

(21) Appl. No.: 15/690,856

(22) Filed: Aug. 30, 2017

Related U.S. Application Data
(60) Provisional application No. 62/381,477, filed on Aug. 30, 2016.

(51) Int. Cl. G01N 21/3577; G01N 21/359; G01N 33/14; G01N 33/04; G01N 33/03

(52) U.S. Cl. CPC G01N 21/3577; G01N 21/359; G01N 33/03; G01N 33/04; G01N 33/146

(57) ABSTRACT

A personal liquid analysis system includes a portable liquid analyzer device, a mobile application as a user interface, and a cloud server for data analysis. The compact and portable liquid analyzer device is integrated by miniaturized near-infrared (NIR) optical sensors setup for spectroscopy measurement from a sample cell, control circuits, I/O user interface modules and wireless communication modules. The analyzer can be used to collect NIR spectra data from a liquid sample in the sample cell, and to transmit the data to a cloud server where compositional information is calculated, and presented to the user on the device and/or on a mobile application. The personal liquid analysis system enables the user to rapidly acquire qualitative and/or quantitative compositional information from liquid samples for a wide range of applications.

### CROSS-REFERENCE TO RELATED APPLICATIONS

The present application claims the benefit of U.S. Provisional Patent Application Ser. No. 62/381,477, which was filed on Aug. 30, 2016, by Qiaochu Li et al. for A PORTABLE PERSONAL BREASTMILK MONITORING SYSTEM, which is hereby incorporated by reference.

### BACKGROUND

### Technical Field

The present invention relates to a liquid analysis system and, more particularly, to a portable liquid analysis system for personal use which automatically and rapidly analyzes the compositional information in liquid samples.

### Background Information

A broad variety of liquids are consumed in people's daily life, such as milk, beverage, alcohol, oil, etc. Since in many scenarios people intake these liquids by drinking, their compositions contain important information that may directly impact a person's life and health, such as calories intake, nutrition content, adulteration, contamination, etc. Nowadays, the increasing consumer interest in personal health has stimulated the emergence of a variety of connected personal health monitoring devices and systems in the market. A personal liquid analysis system that can automatically collect compositional information in liquid samples would be desirable for daily food safety detection and personal health management.

The analysis of liquid composition may be implemented by various types of prior apparatus with different analytical methods. High performance liquid chromatography (HPLC) instruments may identify and quantify multiple components in a liquid sample, but it may require complicated sample preparation, and the data analysis is time-consuming. In comparison, near-infrared (NIR) spectroscopy is a non-invasive analytical method that can rapidly determine the quantity of multiple components in a complex system, which has been utilized in agriculture and food industry. However, these devices are mostly designed for research or industry use, and they are typically too large and too costly for the consumer market. In addition, these analyzers are mostly stand-alone devices, without support from an automatic analysis system that can rapidly translate spectra data to compositional results of interest.

There is therefore needed a personal liquid analysis system that is compact, low-cost and easy to use, which may allow the user to conveniently read out compositional information in liquid samples on a frequent basis.

### SUMMARY

The present invention is a personal liquid analysis system comprising a portable liquid analyzer device for the user to collect composition-related optical spectroscopy data from liquid samples, and an automatic analysis system to analyze the data and present results to the user. This system enables the user to rapidly read out qualitative and/or quantitative compositional information of a liquid sample, such as identification of a sample's category, and/or concentrations of specific analyte species in a sample. Other objects, advantages and novel features of the present invention will become apparent from the following detailed description when considered in conjunction with the accompanying drawings.

### BRIEF DESCRIPTION OF THE DRAWINGS

The various implementations disclosed herein are illustrated by way of example, and not by way of limitation, in the figures of the accompanying drawings, in which like reference numerals may refer to similar elements.

FIG. 1 is a block diagram of an example personal liquid analysis system;

FIG. 2 is a block diagram of an example portable liquid analyzer device;

FIG. 3 is a perspective view of an example portable liquid analyzer device;

FIG. 4A is a schematic cross sectional view of an example sensors setup/liquid sample interface configured for transmission spectroscopy measurement with a broad-band light source and a spectrometer sensor.

FIG. 4B is a schematic cross sectional view of an example sensors setup/liquid sample interface configured for transmission spectroscopy measurement with an LED-array light source and a light intensity detector;

FIGS. 5A and 5B provide two-dimensional and perspective cross sectional views respectively of an example portable liquid analyzer device;

FIG. 6 is a schematic block diagram showing the information flow in the personal liquid analysis system during a measurement process;

FIG. 7 is a flow chart showing example operation steps for the user during a measurement process;

FIG. 8 is a flow chart showing an example work flow of the portable liquid analysis device during a measurement process;

FIG. 9 is a flow chart showing example data analysis procedures for translating raw spectra data to compositional information of the sample;

FIG. 10 is a plot for a set of example absorption spectra data for milk samples with different concentrations, which are collected by the liquid analyzer device and processed by data pretreatment procedures specified in FIG. 9;

FIG. 11 is an exemplary flow chart for the operation of the personal liquid analysis system according to the one or more embodiments described herein wherein the sample is breastmilk;

FIG. 12 is an exemplary flow chart for the operation of the portable personal liquid analyzer system according to the one or more embodiments described herein; and

FIG. 13 an exemplary flow chart for the operation of the portable personal liquid analyzer system according to the one or more embodiments described herein, wherein a user indicates what type of liquid is being analyzed by the system.

### DETAILED DESCRIPTION OF AN ILLUSTRATIVE EMBODIMENT

### Overview of Personal Liquid Analysis System

This disclosure describes a personal liquid analysis system intended to help an individual user rapidly detect compositional information in common liquid samples, including but not limited to, milk, beverage, alcohol, oil, etc. To obtain the information needed, the system uses a compact and portable hardware device referred to as “portable liquid analyzer” to collect optical spectra data which are related to the chemical composition of the sample. The software part of the system, which may include mobile app(s) and back-end programs on the remote cloud server, serves as a management system that controls the operation, performs data analysis, stores the records and displays the results to the user.

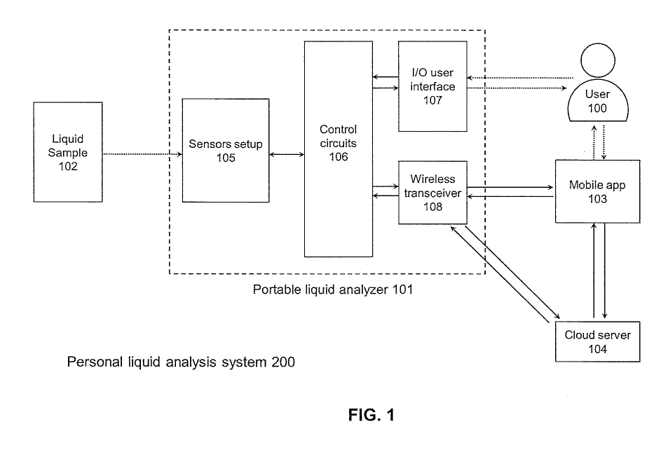

FIG. 1 shows a schematic diagram of an example implementation of a personal liquid analysis system described above. In many embodiments, the personal liquid analysis system 200 comprises a compact and portable liquid analyzer device 101 in wireless communication with a mobile app 103 and a cloud-based server 104. The portable liquid analyzer device 101 may comprise a set of control circuits 106 connected to sensors setup 105, input/output (I/O) user interface devices 107, and one or more wireless transceiver(s) 108 for data collection and transmission.

The control circuits 106 are the control center of the portable liquid analyzer device 101 that handles the device operation, data processing, data storage, and data transmission within internal components as well as to external devices. The control circuits 106 are connected to all the key peripheral components in the analyzer device 101 so that their operations will be controlled by a program stored in the control circuits 106, and the control circuits 106 can directly process and store the data from/to these components.

In many embodiments, sensors setup 105 is connected to the control circuits 106 to collect data from the liquid sample 102. The data of interest include, but are not limited to: information related to chemical composition such as optical spectra, electrode potential, color, etc.; volumetric information such as weight, electric capacitance of the sample, position of sample level, etc.; environmental information such as temperature, humidity, ambient light intensity, etc. Desired information about the liquid sample can be calculated based on the analysis of these obtained data.

In many embodiments, the portable liquid analyzer device 101 comprises one or more wireless transceivers 108 to communicate with external devices through a wireless communication link. In many embodiments, the wireless transceiver 108 can receive commands from and transmit data to the mobile app 103 via wireless protocols including, but not limited to, Bluetooth, Bluetooth Low Energy (BLE), Wi-Fi, near-field communication (NFC) and radio-frequency identification (RFID). In some embodiments, the wireless transceiver 108 can directly exchange data with the cloud server 104 via Wi-Fi connection to the Internet. The wireless transceiver 108 is also connected to the control circuits 106 to transmit external commands to and receive measurement data from the portable liquid analyzer device 101.

In many embodiments, the portable liquid analyzer device 101 comprises a user interface 107 including different input/output (I/O) devices. The user input devices may include but are not limited to buttons, touch screens, voice recognition modules and universal serial bus (USB) ports. These devices allow the user to directly control the operation of the analyzer device 101. The user output devices may include but are not limited to displays, light-emitting diode (LED) signal lights, USB ports. These user output devices can present information to the user including the current status of the analyzer device 101, the analysis results, etc.

In many embodiments, the personal liquid analysis system 200 comprises a mobile application 103 as the major user interface. The mobile app 103 can be based on various devices including smartphones, tablets, PCs and smart watches. Through wireless links including, but not limited to, Bluetooth, BLE, Wi-Fi, NFC and radio-frequency identification RFID, the mobile app 103 can transmit commands to the analyzer device 101 and receive data from it. The mobile app 103 can also send raw measurement data received from the analyzer 101 to the cloud server 104 via Wi-Fi connection to the Internet, and receive analysis results and user's data records. Through these wireless communication links, the mobile app 103 may allow the user to control the analyzer device 101 and view analysis results on the mobile app 103. In some embodiments, the mobile app 103 may provide a record management interface where the user can keep track of the history records. In some embodiments, the mobile app 103 may send notifications such as alerts and/or instructions to the user, based on the analysis results and/or the user's data record.

In many embodiments, the data storage and analysis in the liquid analysis system 200 are performed on the cloud server 104. The cloud server 104 may comprise an analysis center where specific analysis models and algorithms are programmed to process the collected spectra and other measurement data, and calculate the compositional information in the liquid sample. The cloud server may also comprise a user database that stores the measurement data and analysis results of each user as a history record.

The information flow of the liquid analysis system 200 is illustrated by the arrows in FIG. 1 (solid arrows represent information transmitted by electrical signals by circuitry or wireless links, while dash arrows represent information transmitted by non-electrical signals). The user can control the operation of the system 200 on the mobile app 103 or directly on the device 101 by input devices such as buttons and/or touch screens. Once a measurement mode is triggered, the control circuits 106 controls the sensors setup 105 embedded in the analyzer device 101 to collect data from liquid sample. The obtained data is digitized and processed in the control circuits 106, and then transmitted to the mobile app 103 or directly uploaded to the remote cloud server 104. The measurement data received on the mobile app 103 will be subsequently uploaded to the cloud server 104, where the analysis of data and calculation of sample composition is performed. The composition-related results will then be sent back to the mobile app 103 and/or the analyzer device 101 and displayed to the user. The results are also stored in the user database on the cloud server 104 as a history record that the user can check on the analyzer device 101, on the mobile app and/or on a web page.

### Portable Liquid Analyzer Device

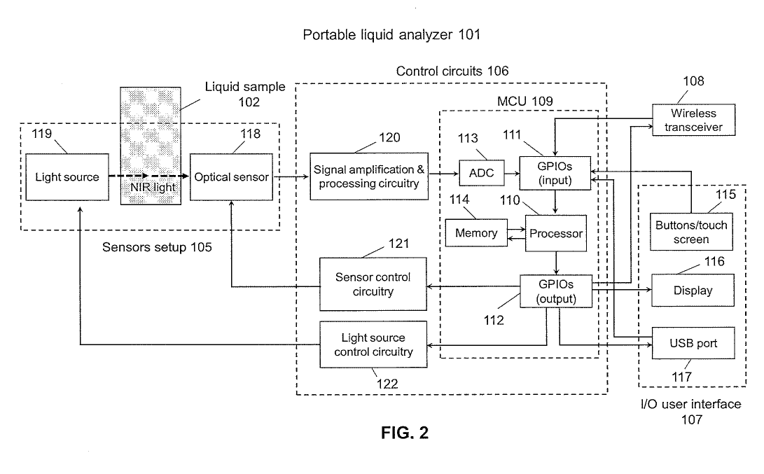

Referring now to the invention in more detail, FIG. 2 depicts a schematic block diagram of an example portable liquid analyzer device 101, as well as the data flow as shown by the arrows. The analyzer device 101 may comprise control circuits 106 connected to sensors setup 105 interfacing the liquid sample 102. Input/output (I/O) devices are also connected to the control circuits 106 as an I/O user interface 107 to exchange data with external environment.

In many embodiments, the control circuits 106 comprises a microcontroller unit (MCU) 109 where data are processed and commands are executed, a signal amplification & processing circuitry 120, a sensor control circuitry 121, and a light source control circuitry 122.

In many embodiments, the MCU 109 comprises a processor 110 for executing calculations and operation tasks. The processor 110 exchange data and commands with peripheral components in the liquid analyzer 101 through general-purpose input/output (GPIO) ports, including GPIOs for input 111 and GPIOs for output 112. In many embodiments, one or more analog-to-digital converters (ADCs) are attached to GPIO input ports 113 for transforming analog readouts from sensors into digital signals that can be processed by the processor 110. In many embodiments, one or more digital-to-analog converters (DACs) are attached to GPIO output ports 112 for transforming digital signal from the processor into analog signals that can modulate the voltage and/or current in the circuitry. In many embodiments, one or more memory units 114 are connected to the processor 110 for storing program commands and data temporarily or permanently. In some embodiments, the memory units 114 comprise volatile memory that requires power to maintain stored information such as random-access memory (RAM). In some embodiments, the memory units comprises non-volatile memory that retains stored information when the power is off, including but not limited to flash memory devices, magnetic disk devices, optical disk drives, magnetic tapes drives. The MCU may be programmed with a set of commands stored in the memory 114, which manages the hardware operation and software workflow.

In some embodiments, the I/O user interface 107 comprises buttons and/or touch screen sensors 115 that functions as the user input devices. These input devices are connected to the GPIO input ports 111 of the MCU 109 so that external information can be input to the analyzer device 101. Users may directly control the operation of the liquid analyzer by inputting commands through these buttons and/or the touch screen.

In some embodiments, the I/O user interface 107 comprises one or more display(s) 116. The display can be of any appropriate type including liquid crystal display (LCD), organic light-emitting diode (OLED) display, cathode ray tube (CRT) monitor, E ink display, etc. The display is connected to the GPIO output ports 112 of the MCU 109 so that it can receive information from the liquid analyzer device. Visual information such as the user interface of the analyzer's operation system and measurement results can be presented to the user on the display.

In some embodiments, the I/O user interface 107 may use a wired connection to charge an internal rechargeable battery and/or exchange data with external devices, through one or more universal serial bus (USB) port(s) 117. The USB port(s) may be connected to the GPIO input ports 111 and output ports 112 of the MCU 109 for data exchange.

In many embodiments, the liquid analyzer device 101 uses the wireless transceiver 108 to communicate with external devices such as smartphones, tablets, smart watches, PCs, cloud server 104, etc., through wireless link including, but not limited to, Bluetooth, BLE, Wi-Fi, NFC and RFID.

In many embodiments, the liquid analyzer device 101 comprises one or more optical sensor(s) 118 in the sensors setup 105. The optical sensor 108 has electrical response upon illumination of near-infrared (NIR) light with wavelength in the range of 700 nm to 2500 nm, which can translate light intensity signals to electrical signals such as voltage or current. In some embodiments, the optical sensor 108 is a spectrometer sensor which can obtain optical spectra data by splitting the incident light based on wavelengths and measuring the incident light intensities at each wavelength. To be embedded in a portable liquid analyzer device, the NIR spectrometer sensor is compact in size, in some embodiments smaller than 4 cm*2 cm*2 cm.

The wavelength scanning mechanism of NIR spectrometer sensor can be of any type, including fixed grating with detector arrays, Fabry-Pdrot interferometer (FPI), scanning grating, interference-filter, Fourier-transform (FT) spectroscopy and any other type known in the art appropriate for spectra measurements. In some embodiments, the optical sensor 108 is a light intensity detector without any wavelength scanning or light splitting mechanisms. It responds to incident light with a broad range of wavelengths. The light intensity sensor can be of any type known in the art, including but not limited to, photodiode, photomultiplier, charge-coupled device (CCD), complementary metal-oxide-semiconductor (CMOS) sensor, etc. To collect spectra data at individual wavelengths, the light intensity detector needs to be coupled with a set of monochromatic light sources, such as an LED array.

In addition to the optical sensor 108, in some embodiments the portable liquid analyzer device 101 may comprise other sensors (not shown) connected to MCU 109 for collection of non-spectroscopic signals from the sample to be analyzed or from environment. These sensors include, but are not limited to, temperature sensors, weight sensors, pH sensors, ion-selective electrodes, cameras or color sensors coupled to chemical test papers, ambient light sensors, GPS units, accelerometers, electrical sensors (e. g., resistance, capacity and inductance), clocks, distance sensors, etc.

In many embodiments, the measured signals from the optical sensor 118 may be first sent to a signal amplification & processing circuitry 120 before transmission to the MCU 109. Since the ADC 113 in the MCU 109 has a limited resolution, amplifying small signals to an appropriate level will enhance the accuracy of measurement data. In addition, some high-frequency noises can be eliminated by processing the signal through a low-pass filter, which may enhance the signal-to-noise ratio. After being processed by the amplifiers and/or filters in the circuitry 120, the analog signals from the sensors are digitized by the ADC 113 in the MCU 109, and the measurement data are subsequently transmitted to external devices such as smartphones, tablets, PCs, smart watches, and the cloud server for further analysis.

In many embodiments, the triggering, wavelength scanning and timing control of the optical sensor 118 is modulated by a control circuitry 121 that is connected to the GPIO output ports 112 of the MCU 109, which may comprise transistors, digital-to-analog converters (DACs), amplifiers and oscillators. When a measurement of one or more sensors need to be triggered at a specific timing, the MCU 109 may send command(s) to the sensor control circuitry 121 to turn the sensor on/off by transistors. Some sensors may comprise their own internal control circuits embedded in the sensor device. In such cases, these internal control circuits can be directly connected to the MCU 109 to receive measurement triggers. For some spectrometer sensors, the wavelength scanning mechanism is modulated by an analog input. In such cases, commands from the MCU 109 will be translated into analog signals for wavelength scanning control by the DAC and amplifiers in the sensor control circuitry 121. In some embodiments, in addition to the MCU's own clock, the sensor control circuitry 121 may comprise one or more external oscillator for the timing control.

In many embodiments, the portable liquid analyzer device 101 comprises a light source 119 together with the optical sensor 118 to provide illumination for optical spectra measurement. As illustrated in FIG. 2, the light source 119 emits light with wavelengths in NIR range (700-2500 nm), which is directed into the liquid sample 102. After traveling through liquid sample, the transmitted light is measured by the optical sensor 118 to generate NIR absorption spectra data of the sample 102. In some embodiments, when a spectrometer sensor is used as the optical sensor 118, the light source provides a broad-spectra illumination. The light source can be of any appropriate type including incandescent lamp (for example tungsten lamp or halogen lamp), gas discharge lamp, light-emitting diode (LED), laser or any combination of them. The wavelength(s), light intensity and power of the light source will depend on the particular configuration of the analyzer device 101.

In some embodiments, when a light intensity detector is used as the optical sensor 118, the light source provides a set of monochromatic light with different wavelengths in NIR range (700-2500 nm). The light source can be an array of monochromatic light emitters with different wavelengths such as LEDs and lasers, or a broad-range light emitter with a tunable monochromatic filter. In some embodiments, the light source may comprise one or more lenses to focus the emission light to the sample. In some embodiments, the light source may comprise a diffuser to scatter the emission light.

In many embodiments, the on/off as well as the illumination intensity of the light source 119 can be controlled by MCU 109 through a light source control circuitry 122 in the control circuits 106 that is connected to the GPIO output ports 112. The control circuitry 122 may comprise DACs to translate digital commands into analog voltage and/or current signals, amplifiers to modulate the input voltage and/or current of the light source, and transistors to switch the light source's power supply on/off.

In some embodiments, the liquid analyzer device 101 may comprise a clock to provide information about current time. The clock may be implemented by an oscillator with a known frequency, together with programs executed by the MCU 109 to generate current time. In some embodiments, the time information may be obtained by synchronizing with the clock on an external device, including but not limited to, smartphones, tablets, PCs, smart watches, and remote cloud servers.

In many embodiments, the liquid analyzer device 101 may comprise a power source. In some embodiments, the analyzer device is powered by one or more rechargeable batteries embedded in the device, such as lithium-ion (Li-ion) battery, lithium-ion polymer (Li-poly) battery, nickel-metal hydride (NiMH) battery, nickel-cadmium (NiCd) battery, lead-acid battery, etc. In such cases, the battery may be connected to a charging circuitry and can be charged through the USB port 117. In some embodiments, the analyzer device 101 is powered by one or more disposable batteries. In some embodiments, the analyzer device 101 is powered by the electrical outlet and/or external devices through a wired power cord.

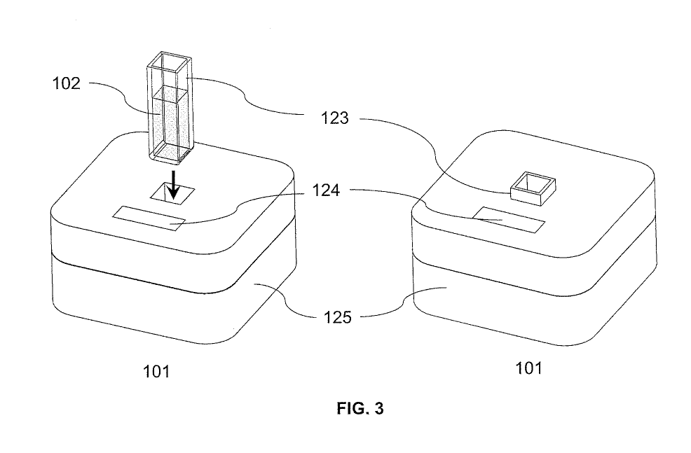

In many embodiments, the components of portable liquid analyzer 101 referred to in the FIG. 2 are integrated in a compact, portable and self-standing device. In some embodiments, the size of the device may be within 15 cm*15 cm*10 cm so that it can be easily carried by the user. Referring to FIG. 3, there is shown an isometric view of an example embodiment of the portable liquid analyzer 101. The control circuits 106, sensors setup 105, batteries and communication modules are embedded inside the device case 125. There is an opening on the top of the device case 125 where a removable sample cell 123 can be plugged in and out. The sample cell 123 is made from materials that are optically transparent in the desired wavelength range, such as quartz, glass, and plastic. The sample cell 123 can be in many different shapes, but the side walls which the NIR light travels through should be transparent and perpendicular to the light path to maximize light transmission. The optical path length of the sample cell 123 may vary according to the device configuration and the exact liquid sample to be analyzed. During a measurement, the liquid sample 102 is contained in the sample cell 123, and the sample cell is plugged into the analyzer device 101 for analysis, as depicted by FIG. 3.

In this and many other embodiments, a control board 124 is located on the surface of the device case 125 as the user interface. The control boards may comprise one or more of the following user input and output devices including buttons, touch screens, displays, LED signal lights, as described in FIG. 1 and FIG. 2. The configuration of components inside the case may vary among different embodiments, according to specific applications.

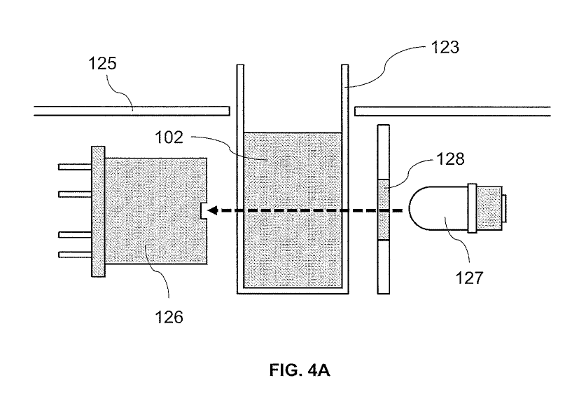

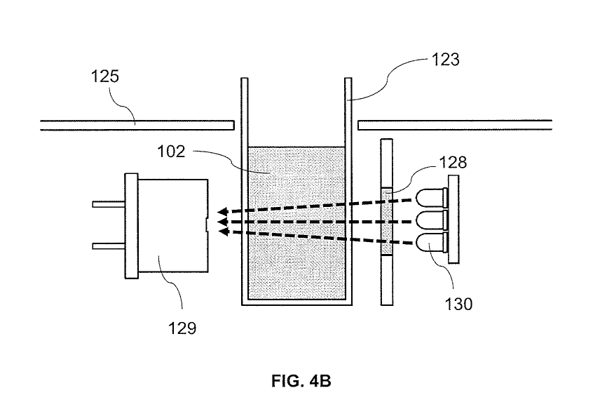

To implement the device configuration for liquid analysis as depicted in FIG. 3, the sensors setup inside the liquid analyzer device are arranged in an order that allows for appropriate interfacing with the liquid sample 102. Referring to FIG. 4A, there is shown a schematic illustration of the configuration of the sensors setup/liquid sample interface in one preferred embodiment. The sample cell 123 is plugged into the liquid analyzer 101 through the openings on the top surface of the device case 125. A spectrometer sensor 126 and a broad-band light source 127 are placed on the opposite sides of the sample cell 123, and the three components are set in a line for transmission spectroscopy measurement. A light slit 128 is placed between the light source 127 and the sample cell 123 to guide the light into the sample. In some embodiments, the slit 128 contains an optical filter to remove light of specific wavelength range, and/or a lens to focus the light. The light emitted by the broad-band light source 127 passes the light slit 128, travels through the liquid sample in the sample cell and enters the slit on the spectrometer sensor 126, as shown by the dash arrow in FIG. 4A. The spectrometer sensor 126 can collect the spectrum data I(λ) of the incident light by reading the light intensity I at each wavelength λ.

In some alternative embodiments, a light intensity detector 129 is used instead of a spectrometer sensor 126. In such case, the broad-band light source 127 is replaced by an array of monochromatic LEDs 130 with different wavelengths in NIR range, as depicted in FIG. 4B. During a measurement, the LEDs 130 with each wavelength λ illuminates in a serial order, and the light intensity detector 129 records the light intensity I at each wavelength λ respectively, as shown in FIG. 4B. In this way, the spectrum data I(λ) of the incident light can also be collected.

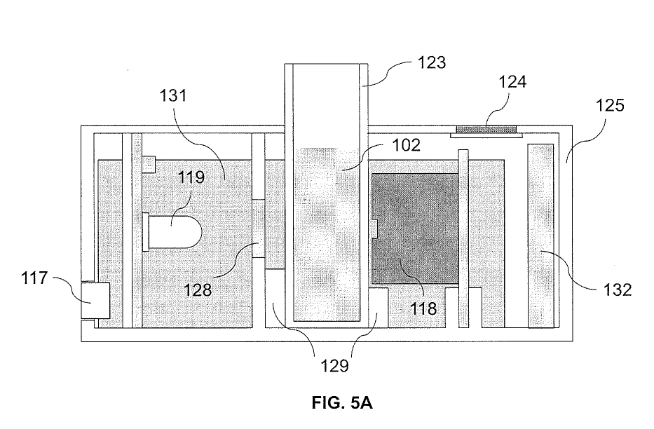

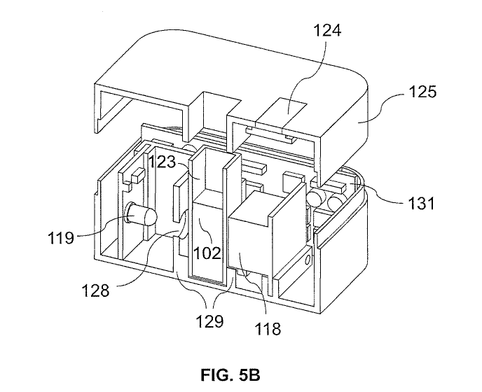

In further details, the internal structure of a portable liquid analyzer device in a preferred embodiment is shown by the cross-sectional schematic illustrations in FIG. 5A and FIG. 5B. In this embodiment, the light slit 128, the optical sensor 118 and the light source 119 alongside with their attached circuit boards are fixed on interior of the device case 125. A sample holder 129 is fixed at the bottom of the device case 125 to secure the position of the sample cell 123 so that it won't move or tilt during measurement. The optical sensor 128 is placed in close proximity to the sample cell 123 to minimize the effect of stray light. The optical sensor 118, the sample holder 129, the light slit 128 and the light source 119 are well aligned so that the optical sensor receives maximum amount of transmission light.

In many embodiments, the control circuits 106 and the wireless transceiver 108 as referred to in FIG. 2 are integrated on one or more printed circuit boards (PCBs) 131, which is included inside the device case 125. In the particular embodiment as depicted in FIG. 5A and FIG. 5B, the PCBs 131 may be fixed in parallel to the side walls of the device case 125. In some other embodiments that are not shown in the figures, the PCBs 131 may be fixed at the bottom of the device case 125. In many embodiments, the PCBs 131 are electrically connected to the light source 119, optical sensor 118, control board 124, and battery unit 132 through electric wires or cables. In many embodiments, the USB port 117 is fixed on the PCB and can be reached from the device exterior through a port opening on the device case 125. An external USB cable can be connected to this port from outside for battery charging and/or data transmission.

In many embodiments, one or more battery units 132 are included in the device as the power supply. In the particular embodiment as depicted in FIG. 5A, the battery units 132 is fixed by the interior side wall of the device case 125. In some embodiments that are not shown in the figures, the battery units 132 may sit at the bottom inside the device case. In some other embodiments, the battery units 132 may be stacked with the PCBs.

In some embodiments, the portable liquid analyzer 101 disclosed herein may be implemented as a stand-alone device. In some other embodiments, the liquid analyzer 101 may be implemented to leverage external devices for certain comprehensive applications, through wired and/or wireless connections. The external devices include, but are not limited to, external sensors, heaters, stirrers, pumps (e.g. breastmilk pump), scales, smart watches, sphygmomanometers, wearable biometric monitoring devices.

### Data Collection

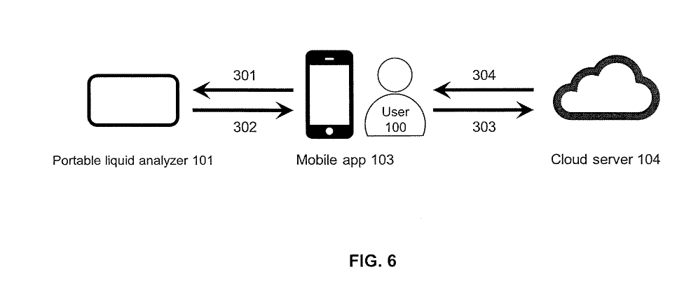

In many embodiments, a measurement process, which is a working cycle in the liquid analysis system 200 to determine the composition-related information of the sample such concentrations of analyte species and/or classification of the sample, involves information flow among the portable liquid analyzer device 101, the mobile app 103 and the cloud server 104, as illustrated in FIG. 6. In a preferred embodiment, the information flow follows a sequential order: (1) the user 100 starts a measurement event on the mobile app 103 or directly on the portable liquid analyzer 101 by I/O user interface 107, and the command is sent to the analyzer device (step 301); (2) the analyzer 101 collects spectra data from the liquid sample to be analyzed, and the data are sent back to the mobile app 103 (step 302); (3) the raw spectra data are uploaded to the cloud server 104, and processed by an analysis algorithm; (4) after being analyzed on the cloud server 104, the raw spectra data are translated into composition-related information, and the results are sent back to the mobile app and presented to the user (step 304).

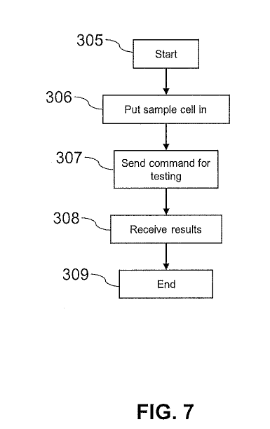

The design of this personal liquid analysis system 200 allows a user to quickly get compositional information of a liquid sample with minimal efforts. In many embodiments, the user can complete a measurement process by following a simple set of operation procedures as illustrated in FIG. 7. To start a measurement, the user first puts a sample cell 123 that contains the liquid sample to be analyzed into the portable liquid analyzer device 101 (step 306). Then the user sends a “start testing” command to the analyzer device 101 (step 307). The data collection and analysis processes will then be automatically executed by the analyzer device 101 and the cloud server 104, and the user will receive analysis results on the mobile app 103 (step 308).

In some embodiments, the user's commands in step 307 may be input directly on the analyzer device 101 by user input devices such as buttons and/or touch screens. In some embodiments, the command may be input from a mobile app 103 and sent to the analyzer 101 through wireless communication.

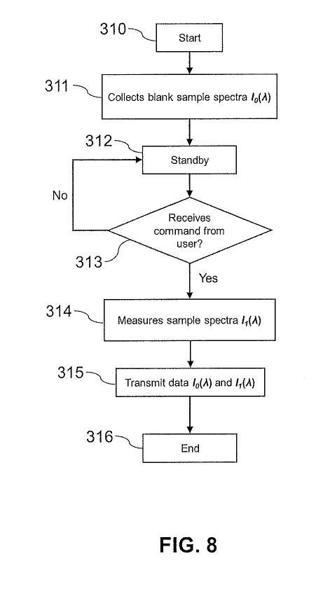

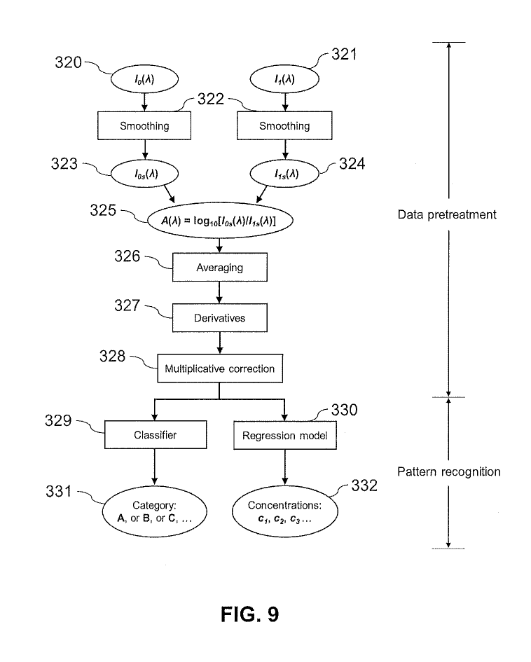

In many embodiments, the workflow of the portable liquid analyzer 101 is pre-programmed with executive instructions to accomplish data collection and transmission tasks. In further details, reference is now made to FIG. 8 which shows an example workflow of the liquid analyzer device 101 for data collection in “manual mode”, and FIG. 9A which shows the user's operation procedures accordingly. Before a sample is added to the device 101 for analysis, the analyzer device 101 may first collect the blank background spectra I₀(λ) (detected light intensity I₀ as a function of wavelength λ, when no sample is present) in the step 311. Then the analyzer 101 is in standby mode 312 waiting for user's command. Once a “start testing” command sent in step 307 specified in FIG. 7 is received, the analyzer device 101 will trigger the measurement event 314, in which the analyzer 101 measures the sample spectra I₁(λ). The collected I₀(λ) and I₁(λ) data in step 311 and step 314 are then transmitted to a mobile app 103 and/or a remote cloud server 104 in following step 315.

### Data Analysis

The overtone and combinational vibrations of a molecule can be excited by electromagnetic waves in NIR region. Therefore, the NIR absorption spectra contains rich information about the system's chemical composition. In the liquid analysis system disclosed herein, the spectra data collected from the portable liquid analyzer device are eventually uploaded to the cloud server 104, where various processing and calculation procedures are performed on the data to generate composition-related information for the user, such as concentrations of one or more analyte species, and/or classification of the liquid sample 102. The data analysis programs implemented on the cloud server enables rapid and automatic analysis of the composition in the liquid sample 102 of interest for the user. In further details, the data analysis programs usually include 2 major parts: data pretreatment and pattern recognition by machine learning.

### 1. Data Pretreatment

Referring to FIG. 9, there is shown an example flow chart of the analysis procedures of a sample's NIR spectra data. In many embodiments, once the raw spectra data (I₀(λ) 320 and I₁(λ) 321) collected in step 311 and 314 as specified in FIG. 8 have been uploaded to the cloud server 104, a local convolution filter will be firstly applied to the data that acts as a smoothing method 322. The objective of smoothing spectral data is the reduction of noise, which can be described as random high-frequency perturbations. One or more smoothing methods may be employed including, but not limited to, the simple filter coefficient vector for the moving average method, the Savitzky-Golay method, optimal Wiener filter, adaptive smoothing method by taking into account the local statistics of the observed waveform. From this step, the output would be the spectra data after the reduction of noise, specified as I₀ₛ(λ) 323 and I₁ₛ(λ) 324 respectively.

The following step will be translating the spectra data (I₀ₛ(λ) and I₁ₛ(λ)) to the optical absorbance. Based on Beer-Lambert's law, the optical absorbance of a substance Aᵢ is proportional to its concentration cᵢ. Therefore, for quantification of the chemical composition in the sample, the spectra data I₀(λ) and I₁ₛ(λ) can be transformed into absorbance 325: A(λ)=log₁₀ [I₀ₛ(λ)/I₁ₛ(λ)].

In many embodiments, multiple measurements are conducted in the same measurement process to further suppress signal noise. Following the same procedure, a set of parallel absorption spectra, A₁(λ), A₂(λ), A₃(λ), A₄(λ), A₅(λ), . . . , are collected during a same measurement process and are used to get the average absorption spectra A_{avg}(λ) in step 326. In many embodiments, derivatives of the spectra data are calculated in the following step 327 to remove or suppress constant background signals and to enhance the visual resolution. Background signals and global baseline variations are low-frequency phenomena, so derivatives can be interpreted as high-pass filters. Since each derivative reduces the polynomial order by one, a constant offset is removed. In spectroscopic applications, different order derivatives may be used including, 1^{st} derivatives (It transforms the linear term into a constant one, thus removing linear tilting of the graph), 2^{nd} derivatives (It transfers peak maxima into minima and vice versa). This is a valuable tool for identifying weak peaks that are not visible in the original spectrum.

In some embodiments and/or applications, the liquid samples 102 to be analyzed are opaque suspension or emulsion with significant light scattering, such as milk, juice, etc. The light scattering may result in random variation in optical path length, which creates difficulties for building an accurate and consistent analysis model in the successive data pattern recognition process. In such cases, a data pretreatment step 328 is needed to correct these multiplicative effects. Several known multiplicative correction methods can be used to minimize the spectra deviation caused by light scattering, including but not limited to simple 1-Norm normalization, multiplicative scatter correction (MSC) and standard normal variate (SNV) method.

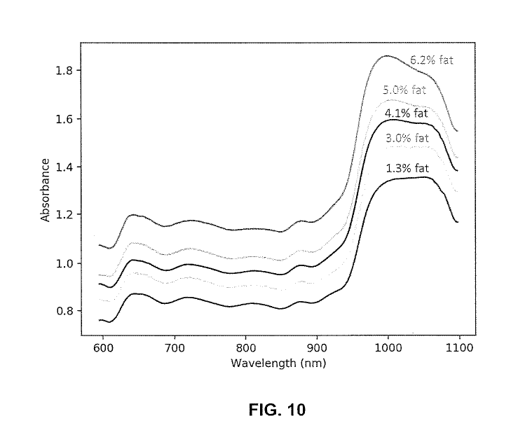

Referring to FIG. 10, there is shown an example set of absorption spectra plots for milk samples with different fat concentrations, which are collected by the portable analyzer device and processed by data pretreatment methods specified above.

### 2. Pattern Recognition (by Machine Learning)

After data pretreatment, the sample spectra data can be then used as the testing data and/or the training set for the pattern recognition algorithm based on machine learning, which translates the sample's spectral data to compositional information. According to the specific applications to be implemented, the pattern recognition tasks may include two major categories: classification and regression.

In some embodiments and/or applications, the task to be performed is qualitatively recognizing the category of the sample, which is defined as a classification task. For example, in some embodiments and/or applications, the portable liquid analysis system disclosed herein is used to determine the specific category or brand of a wine. The objective of this problem is to find a classifier 329 by learning from a given set of database (also known as training set), so that the classifier can directly predict (classify) an output (brand of a wine) from an unseen input (new sample spectra). In this case, the database is sets of spectra (input) and brand of the wine (output) pairs.

A classification algorithm can be used to approach this problem including, but not limited to, support vector machine (SVM), logistic regression, and neural networks. One possible detailed example task is that a chosen classification algorithm is trained by ‘learning’ the given training set, spectra 1—brand A wine, spectra set 2—brand B wine, spectra set 3—brand C wine, etc., to find the optimal classifier. Therefore, when an unseen (wine) spectra is measured from a random brand wine (note that the brand has to be seen in the training set), this classifier can directly predict the results 331—brand of this wine (e.g. brand B wine). Based on the specific classifier trained by different training sets in the database, the portable liquid analysis system can be used to conduct different classification tasks for different types of samples, without changing the hardware device setup.

In some embodiments, the task to be performed is quantitatively calculating the concentration of one or more analyte components in the sample, which is defined as a regression task. For example, in some embodiments and/or applications, the portable liquid analysis system disclosed herein is used to determine the concentrations of macronutrients in milk including fat, protein, carbohydrate, etc. For the quantitative analysis for the spectra data in these systems, multivariate linear regression methods may be used to build the regression model 330, such as principal component regression (PCR) and partial least squares (PLS) regression. In some embodiments, non-linear multivariate calibration techniques such as artificial neuron network and genetic algorithm may be used.

To build the regression model for the subject analysis, the spectra data of a series of samples with known output vector (the analyte concentrations) are collected on the analyzer as the training set. To further formalize the learning problem, a self-defined objective function (e.g. ordinary least squares (OLS) or linear list squares) is needed, so the goal of the learning model will be minimizing objective function through the “learning process” (iteration). Different optimization methods for minimizing an objective function can be selected including, but not limited to, stochastic gradient descent (SGD), analytical approach. Multivariate regression models (e.g. neural networks) can be performed based on the database to find the optimal model. Then a self-defined metrics function is used to determine performance of each model generated from different architectures (e.g. neural networks, PCR, linear regression, non-linear regression (Kernels)), so to select the final prediction model for the problem. With this model, the predicted concentrations results 332 of each analyte component (c₁, c₂, c₃, . . . , cₙ) in a sample can be back-calculated from its spectra data A(λ). Based on the specific prediction model trained by different training sets in the database, the portable liquid analysis system can be used to conduct different regression tasks for different types of samples, without changing the hardware device setup.

### Applications

The liquid analyzer device disclosed herein may be used as a portable device that can provide rich information on a liquid sample's composition. Because of the device's compact size, easy operation, no need for sample pretreatment and built-in automatic analysis function, the personal liquid analysis system disclosed herein is particularly suitable for personal and/or family daily use as a consumer product. With the features and functions disclosed herein, this personal liquid analysis system can be used for acquiring qualitative and/or quantitative composition-related information for a wide range of liquid samples and applications.

For example, the personal liquid analysis system disclosed herein may be used to determine the concentrations of multiple nutrients simultaneously in a milk sample. Here “milk” refers to a wide range of diary liquid including but not limited to, cow's milk (with different fat levels), sheep's milk, human breastmilk, formula milk, milk drinks, drinkable yogurt, etc. The nutrient components that can be analyzed may include, but are not limited to, lactose, proteins (casein, whey protein, total protein), fat, fatty acids, vitamins, and mineral ions (calcium, sodium, potassium, etc.). These results can be calculated by the regression method disclosed in the “Data analysis” section. Total calories in the milk sample can be calculated based on the concentration of nutrients with publicly available nutrient calories data. With the nutrient content information, the users can quantify and track the nutrition intake from the milk consumed for their nutrition and health management.

In another example, the personal liquid analysis system disclosed herein may also be used to determine the sugar concentration in beverages, including but not limited to, carbonated drink, juice, tea drink, coffee, etc. These results can be calculated by the regression method disclosed in the “Data analysis” section. Total calories in the beverage can be calculated based on the concentration of sugar. The sugar level and contained calories provides important health information for those who are concerned, such as people keeping a diet, people with hyperglycemia and diabetes patients.

In another example, the personal liquid analysis system disclosed herein may also be used to determine the alcohol percentage in alcoholic drinks, including but not limited to, liquor, wine, beer, sake, cocktail, etc. These results can be calculated by the regression method disclosed in the “Data analysis” section. Based on the alcohol percentage in the drink, total alcohol intake can be calculated as important health information.

In another example, the personal liquid analysis system disclosed herein may also be used to identify alcoholic drinks with category, brand, quality, etc. These results can be determined by the classification method disclosed in the “Data analysis” section. These types of information can be used to evaluate the quality and value, verify the brand, and/or determine genuineness for alcoholic drinks.

In another example, the personal liquid analysis system disclosed herein may also be used to identify adulteration in liquid products, such as milk with added melamine, recycled oil, alcoholic drinks blended with industrial alcohol, etc. These results can be determined by the classification method disclosed in the “Data analysis” section so that the samples with adulteration can be distinguished from normal ones. Such information can be very important for portable quality screening and personal food safety management.

In another example, the personal liquid analysis system disclosed herein may also be used to identify substances in liquid that are harmful to human health, including but not limited to, toxic substances, carcinogen, allergen, etc. Depending on the specific analyte and application, analysis results may include whether the analyte is detected (determined by the classification method disclosed in the “Data analysis” section), and/or the quantitative amount of analyte in the sample (determined by the regression method disclosed in the “Data analysis” section). Such information is critically important for applications such as environment monitoring, allergy prevention and personal food safety management.

In some applications, the personal liquid analysis system disclosed herein may be integrated with a software management system. Such management system may track and record the analysis results generated by the personal liquid analysis system and store them in a user database, which allows each individual user to monitor compositional information of interest in a continuous period of time, and recall history records. In some applications, such management system may generate executable instructions and/or recommendations to the user based on the analysis results from the liquid analysis system. For example, when the personal liquid analysis system disclosed herein is used for nutrition analysis for human breastmilk, a management system can track the amount of the baby's daily nutritional and caloric intake. Based on these data, the system can provide mothers with instructions on appropriate feeding amount and timing, as well as recommendations of food to keep breastmilk nutrition balanced and healthy.

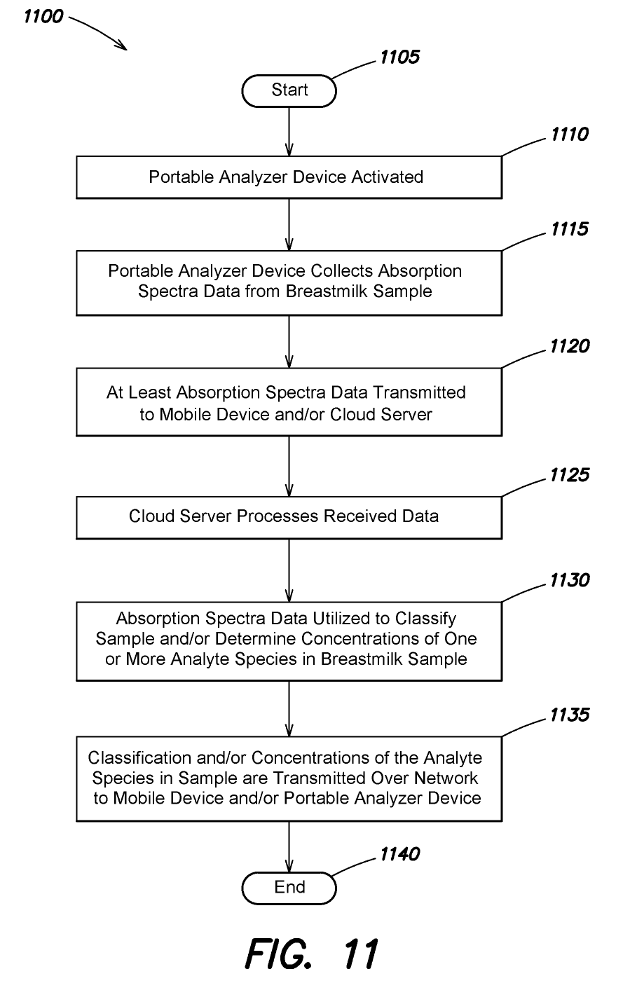

FIG. 11 is an exemplary flow chart for the operation of the personal liquid analysis system according to the one or more embodiments described herein wherein the sample is breastmilk. The procedure 1100 starts at step 1105 and continues to steps 1110 where the portable analyzer device is activated. The user may first power on the portable analyzer device 101 in a variety of different ways. For example, the user may select a “power button” from within the mobile application 103 executing on the mobile device to send a signal over the network to the portable analyzer device 101 that powers on the portable analyzer device 101. Alternatively, the user may press a “power button” on the portable analyzer device 101 to power on the portable analyzer device 101.

Once the sample 102 is in the sample cell 123 and ready to be analyzed, the user may, for example, utilize the mobile application 103 executing on the mobile device to begin the analysis of the sample 102. Specifically, the user may select a “start” button from within the application 103 to begin the analysis of the sample 102. The selection of the “start” button may cause a signal to be transmitted over the network from the mobile device to the MCU 109 of the portable analyzer device 101 indicating that the analysis of the sample 102 should begin. Alternatively, the user may select one or more buttons on the I/O user interface 107 of the portable analyzer device 101 to indicate that analysis of the sample 102 should begin. Activation of the portable analyzer device 101 includes, but is not limited to, providing power to the light source 119 and the sensor 118 such that they are turned on and in “measurement mode.” It is noted that before the sample 102 is placed within the sample cell 123, the portable analyzer device 101 may first collect blank background spectra data (e.g., when no sample 102 is within the sample cell 123 or a liquid, e.g., deionized water, which does not contain any other analytes is within the sample cell 123).

The procedure continues to step 1115 and the portable analyzer device collects absorption spectra data from the breastmilk sample. Specifically, the light source 119 provides broad-spectra illumination or a set of distinct wavelength of monochromatic light. The incident light is directed into the sample and after absorption by the breastmilk sample, the transmitted or scattered light is measured by the optical sensor 118 (e.g., near-infrared (NIR) spectroscopy sensor) of the portable analyzer device 101 to collect absorption spectra data. It is noted that other sensors, such as a temperature sensor and a weight sensor, as described above, may be utilized to collect other data. For example, the collected data may include, but is not limited to, chemical composition information such as optical spectra, electrode potential, color; volumetric information such as weight, electric capacitance of the sample, position of sample level; environmental information such as temperature, humidity, ambient light intensity. It is noted that the portable analyzer device 101 may digitize the collected data for transmission over the network.

The procedure continues to step 1120 and at least the absorption spectra data is transmitted to the mobile device and/or cloud server 104. The absorption spectra data and other data (e.g., the other collected data and blank background spectra data) may be transmitted over the network to the mobile device and/or cloud server 104 for storage. If the portable analyzer device 101 is unable to transmit the absorption spectra data to the cloud server 104 due to a network connection issue, or for any of a variety of other reasons, the mobile device may transmit the absorption spectra data to the cloud server 104. Specifically, the user may utilize the application 103 to send the absorption spectra data via a Wi-Fi connection or cellular connection to the cloud server 104. It is noted that after the collected data has been transmitted to the mobile device and/or cloud server 104, the user may turn off the portable analyzer device 101. Specifically, the user may turn off the portable analyzer device 101 in a manner similar as to how the portable analyzer device 101 is turned on, as described above.

The procedure continues to step 1125 and the cloud server 104 processes the received data. Specifically, the absorption spectra data may first be preprocessed to remove spectral variation related to sample and instrument variation. For example, multiple preprocessing techniques may be employed including, but not limited to, filtering, smoothing, spectral derivatives, baseline correction (using the blank background spectra data), multiplicative corrections and standardization, which are understood by those skilled in the art and as described above with respect to FIG. 9.

The procedure continues to step 1130 and the absorption spectra data is utilized to classify the sample and/or determine the concentrations of one or more analyte species in the breastmilk sample.

For example, and as describe above with reference to FIG. 9, one or more analysis models (e.g., regression models) may be created prior to implementation of the personal liquid analysis system. Utilizing the previously created analysis model, the sample may be classified and/or the concentrations of each analyte species in the sample being may be determined. For the breastmilk sample, for example, the analyte species may be, but are not limited to sugars (e.g., lactose), proteins, fat, fatty acids, vitamins, hormones, and mineral ions (e.g., calcium, sodium, and potassium). In addition, it is noted that the previously created analysis models may be refined or updated based on the received absorption spectral data, such that the analysis of future samples maybe more accurate using the refined analysis model.

The procedure continues to step 1135 and the classification and/or concentrations of the analyte species in the sample are transmitted over the network to the mobile device and/or portable analyzer device 101. Specifically, the determined classification and/or concentrations of the one or more analyte species may be transmitted to the mobile device such that the classification and/or concentrations are displayed in the mobile application 103 for the user to view. In addition to or alternatively, the classification and/or concentrations may be displayed on the I/O user interface 107 of the portable analyzer device 101. The procedure then ends at step 1140.

Although reference is made to analyzing a breastmilk sample, it is expressly contemplated that any of a variety of different liquids may be analyzed in a similar manner as described above. For example, a beverage, such as wine and soda may be analyzed in a similar manner as described above. Specifically, the absorption spectra data associated with the beverage would be obtained in a similar manner as described above, and the cloud server 104 would classify the beverage and/or determine the concentration of the analyte species in the beverage.

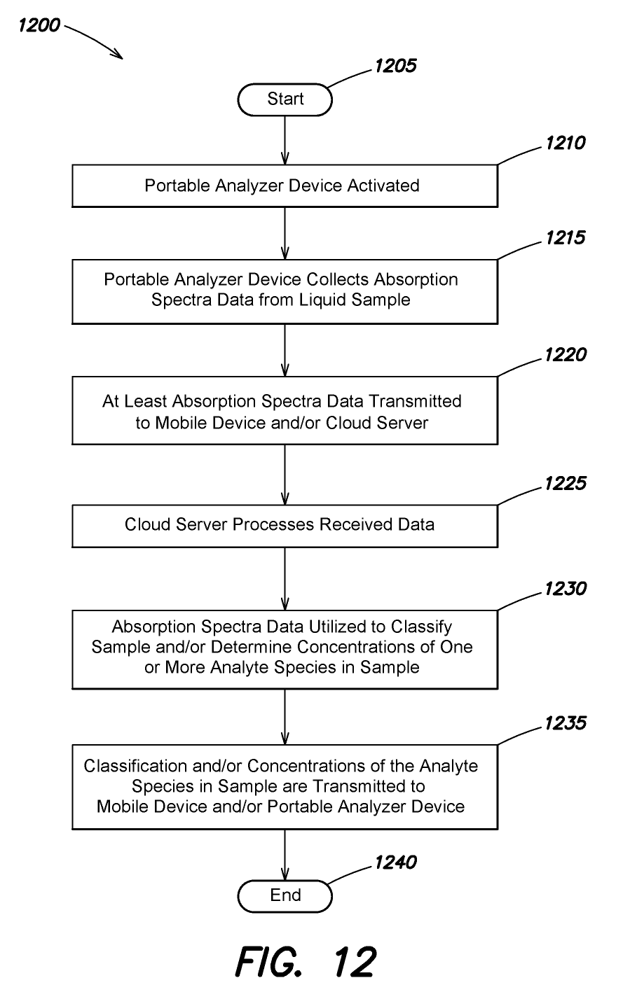

FIG. 12 is an exemplary flow chart for the operation of the portable personal liquid analyzer system according to the one or more embodiments described herein. The procedure 1200 starts at step 1205 and continues to steps 1210 where the portable analyzer device 101 is activated. The user may first power on the portable analyzer device 101 in a variety of different ways. In addition, the user may provide the liquid sample 102 within the sample cell 123. For example, the liquid may be, but is not limited to, beverages (e.g., wine, soda, and coffee), bodily fluids, and oils.

Once the liquid sample 102 is ready to be analyzed, the user may start the analysis in a manner similar to that described above with reference to FIG. 11. Activation of the portable analyzer device 101 includes, but is not limited to, providing power to the light source 119 and the optical sensor 118 such that they are turned on and in “measurement mode.” It is noted that before the liquid sample 102 is placed within the sample cell 123, the portable analyzer device 101 may first collect blank background spectra data ((e.g., when no sample 102 is within the sample cell 123 or a liquid, e.g., deionized water, which does not contain any other analytes is within the sample cell 123).

The procedure continues to step 1215 and the portable analyzer device 101 collects absorption spectra data from the liquid sample. It is noted that other sensors, such as a temperature sensor and a weight sensor, as described above, may be utilized to collect other data.

The procedure continues to step 1220 and at least the absorption spectra data are transmitted to the mobile device and/or cloud server 104. The absorption spectra data and other data (e.g., the other collected data and blank background spectra data) may be transmitted over the network to the mobile device and/or cloud server 104 for storage. If the portable analyzer device 101 is unable to transmit the sample spectra data to the cloud server 104 due to a network connection issue, or for any of a variety of other reasons, the mobile device may transmit the received sample spectra data to the cloud server 104.

The procedure continues to step 1225 and the cloud server 104 processes the received data. Specifically, the sample spectra data may first be preprocessed to remove spectral variation related to sample and instrument variation. The procedure continues to step 1230 and the absorption spectra data is utilize to classify the sample and/or determine the concentrations of one or more analytes species in the liquid sample.

The procedure continues to step 1235 and the classification and/or determined to concentrations are transmitted to the mobile device and/or analyzer device. Specifically, the classification and/or concentrations may be transmitted to the mobile device such that the classification and/or concentrations are displayed in the mobile app 103 for the user to view. In addition to or alternatively, the classification and/or concentrations may be displayed on I/O interface 107 of the portable analyzer device 101. The procedure then ends at step 1240.

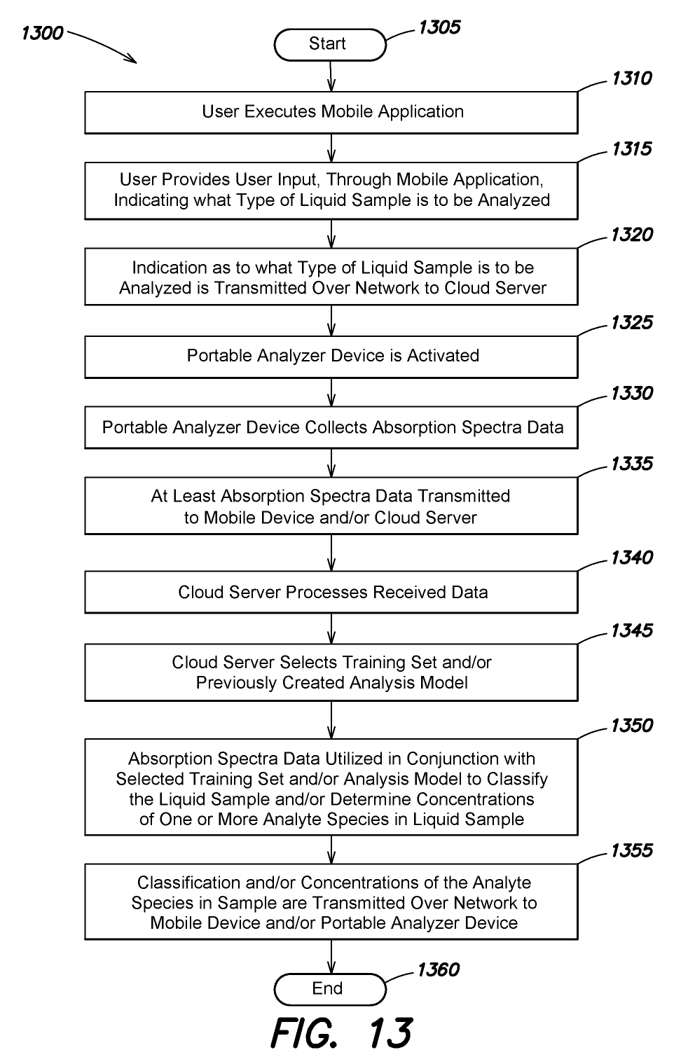

FIG. 13 an exemplary flow chart for the operation of the portable personal liquid analyzer system according to the one or more embodiments described herein, wherein a user indicates what type of liquid is being analyzed by the system. The procedure 1300 starts at step 1305 and continues to steps 1310 where a user executes the mobile application associated with the portable personal fluid analyzer system 200. Specifically, the user may select an icon associate with the application on the mobile device.

The procedure continues to step 1315 and user provides user input, through the mobile application, indicating what type of liquid sample is to be analyzed. For example, the user may want to analyze a sample of breastmilk. Alternatively, the user may want to analyze a beverage sample, such as wine or soda. The indication provided by the user corresponds to the liquid sample that the user will place in the sample cell 123 for analysis. For example, the user may be provided with a drop-down menu from within the mobile application and the user may select a particular type of liquid provided in the drop-down menu. The procedure continues to step 1320 and the indication provided by the user as to what type of liquid sample is to be analyzed is transmitted over the network to the cloud server 104. The procedure continues to step 1325 and the portable analyzer device 101 is activated. Specifically, the portable analyzer device 101 is activated in a similar manner as described above with reference to FIGS. 11 and 12.

The procedure continues to step 1330 and the portable analyzer device 101 collects absorption spectra data from the liquid sample. Specifically, the light source 119 emits light and the optical sensor 118 (e.g., NIR spectroscopy sensor) measures the transmitted and scattered light to collect the absorption spectra data. It is noted that other sensors, such as a temperature sensor and a weight sensor, as described above, may be utilized to collect other data.

The procedure continues to step 1335 and at least the absorption spectra data is transmitted to the mobile device and/or cloud server 104. The sample spectra data and other data (e.g., the other collected data and blank background spectra data) may be transmitted over the network to the mobile device and/or cloud server 104 for storage.

The procedure continues to step 1340 and the cloud server 104 processes the received data. Specifically, the absorption spectra data may first be preprocessed. The procedure continues to step 1345 and the cloud server 104 selects a training set and/or a previously created analysis model based on the user input indicating the type of liquid being analyzed. For example, if the user indicated that breastmilk is to be analyzed, a training set associated with breastmilk and/or a previously created breastmilk analysis model is selected. Alternatively, if the user indicates that soda is to be analyzed, a training set associated with soda and/or a previously created soda analysis model is selected. It is noted that the previously created analysis model may be refined or updated based on the received absorption spectral data, such that the analysis of future samples maybe more accurate using the refined calibration model.

The procedure continues to step 1350 and the absorption spectra data is utilized in conjunction with the selected training set and/or analysis model to classify the liquid sample and/or determine the concentrations of one or more analyte species in the liquid sample. For example, if the liquid sample being analyzed is wine, the cloud server 104 may select a training set for wine that indicates that first spectra data is associated with Pinot Noir, second spectra data is associated with the Malbec, and third spectra data is associated with Cabernet Sauvignon. In addition to or alternatively, the cloud server 104 may select an analysis model associated with wine to determine the concentration of one or more analyte species in the sample based on a comparison of the absorption spectra data and the analysis model.

The procedure continues to step 1355 and the classification and/or concentrations may be transmitted to the mobile device and/or portable analyzer device 101. As such, that the classification and/or concentrations may be displayed within the mobile app 103 for the user to view. In addition or alternatively, the classification and/or concentrations may be displayed on I/O user interface 107 of the portable analyzer device 101. The procedure then ends at step 1360.

What is claimed is:

1. A liquid analysis system, comprising:
a portable liquid analyzer including a control circuit configured to operate a light source and an optical sensor;
the light source configured to emit light, wherein the emitted light travels through a sample;
the optical sensor configured to measure transmitted light from the sample and generate near-infrared (NIR) absorption spectra data of the sample, the sample being a particular type of liquid;
the portable liquid analyzer configured to transmit, over a network, the NIR absorption spectra data to a cloud server;
the cloud server configured to:
classify the sample as a particular variety of a plurality of different varieties of the type of liquid based on utilization of the absorption spectra data in conjunction with a training set that defines the plurality of different varieties of the type of liquid, or
determine a concentration of one or more analytes in the sample based on utilization of the absorption spectra data with an analysis model;
the cloud server further configured to:
transmit, over the network, at least one of the classification and the determined concentrations to a mobile application executing on a mobile device.

2. The system of claim 1, wherein the sample is a homogeneous transparent liquid or a translucent liquid.

3. The system of claim 1, wherein the mobile application receives input to send a signal over the network to the portable liquid analyzer to power on the portable liquid analyzer.

4. The system of claim 1, wherein
the one or more analytes include one or more of: a carbohydrate, a protein, a fat, a fatty acid, a vitamin, a hormone, and a mineral ion, and
the sample is one of milk, an alcoholic beverage, a non-alcoholic beverage, yogurt, bodily fluid, and an oil.

5. The system of claim 1, wherein the light source provides broad-spectra illumination or a set of monochromatic light with different wavelengths.

6. The system of claim 1, wherein the portable liquid analyzer is further configured to generate background spectra data and transmit the background spectra data to the cloud server over the network, and wherein the cloud server is further configured to correct the absorption spectra data using the background spectra data.

7. The system of claim 6, wherein the cloud server is configured to implement one or more smoothing techniques to reduce noise in the background spectra data and the absorption spectra data.

8. The system of claim 1, wherein the analysis model is refined utilizing the absorption spectra data.

9. The system of claim 1, wherein the analysis model is a regression model.

10. A liquid analysis system, comprising:
a mobile application executing on executing on a mobile device, the mobile application configured to:
receive one or more first input commands selecting a type of liquid, of a plurality of different types of liquid, to be analyzed,
transmit, over a network, the selection of the type of liquid to be analyzed a cloud server,
receive one or more second input commands to power on a portable liquid analyzer, and
transmit, over the network, a signal to the portable liquid analyzer indicating that the portable liquid analyzer should be powered on;
the portable liquid analyzer including a control circuit configured to operate a light source and an optical sensor;
the light source configured to emit light in response to the portable liquid analyzer receiving the signal, wherein the emitted light travels through a sample;
the optical sensor configured to measure transmitted light from the sample and generate near-infrared (NIR) absorption spectra data of the sample;
the portable liquid analyzer configured to transmit, over a network, the absorption spectra data to the cloud server;
the cloud server configured to:
select analysis model of a plurality of different analysis models based on the selection of the type of liquid to be analyzed or select a training set of a plurality of different training sets based on the selection of the type of liquid to be analyzed;
the cloud server further configured to:
classify the sample as a particular variety of a plurality of different varieties of the type of liquid based on utilization of the absorption spectra data in conjunction with the selected training set that defines the plurality of different varieties of the type of liquid, or
determine a concentration of one or more analytes in the sample based on utilization of the absorption spectra data with the selected analysis model; and
the cloud server further configured to:
transmit, over the network, at least one of the classification and the determined concentrations to the mobile application executing on a mobile device

11. The system of claim 10, wherein the sample is one of: milk, an alcoholic beverage, a non-alcoholic beverage, yogurt, bodily fluid, and an oil.

12. The system of claim 10, wherein the one or more analytes include one or more of: a carbohydrate, a protein, a fat, a fatty acid, a vitamin, a hormone, and a mineral ion.

13. The system of claim 10, wherein the light source provides broad-spectra illumination or a set of monochromatic light with different wavelengths.

14. The system of claim 10, wherein the portable liquid analyzer is further configured to generate background spectra data and transmit the background spectra data to the cloud server over the network, and wherein the cloud server is further configured to correct the and absorption spectra data using the background spectra data.

15. The system of claim 14, wherein the cloud server is configured to implement one or more smoothing techniques to reduce noise in the background spectra data and the absorption spectra data.

16. The system of claim 10, wherein the analysis model is refined utilizing the absorption spectra data.

17. The system of claim 10, wherein the analysis model is a regression model.

18. A method comprising:
generating near-infrared (NIR) sample spectra data of a sample contained within a portable liquid analyzer, the sample being a type of liquid;
transmitting, over a network, the NIR absorption spectra data to a cloud server;
classifying the sample as a particular variety of a plurality of different varieties of the type of liquid based on utilization of the absorption spectra data in conjunction with a training set that defines the plurality of different varieties of the type liquid; and
determining a concentration of one or more analytes in the sample based on utilization of the absorption spectra data with a regression model, and
transmitting, over a network, at least one of the classification and the determined concentrations to a mobile application executing on a mobile device.

19. The method of claim 18, wherein the sample is one of: milk, an alcoholic beverage, a non-alcoholic beverage, yogurt, bodily fluid, and an oil.

20. The method of claim 18, wherein the one or more analytes include one or more of: a carbohydrate, a protein, a fat, a fatty acid, a vitamin, a hormone, and a mineral ion.
